(19) United States
(12) Patent Application Publication
(10) Pub. No.: US 2022/0401495 A1
(43) Pub. Date: Dec. 22, 2022

(54) ACELLULAR PLACENTAL THERAPIES FOR NECROTIZING ENTEROCOLITIS

(71) Applicant: PLAKOUS THERAPEUTICS, INC., Winston-Salem, NC (US)

(72) Inventors: Seth Tomblyn, Winston-Salem, NC (US); Scott A. Washburn, Winston-Salem, NC (US)

(21) Appl. No.: 17/780,173

(22) PCT Filed: Nov. 27, 2020
(86) PCT No.: PCT/US2020/062475

§ 371 Date: May 26, 2022

Related U.S. Application Data
(60) Provisional application No. 62/941,382, filed on Nov. 27, 2019.

(51) Int. Cl. A61K 35/50; A61K 9/08; A61K 9/00; A61P 1/00; A61K 45/06; A61K 31/145; A61K 38/57; A61K 38/55; A61K 33/02; A61K 31/198; A61K 31/10; A61K 31/40; A61K 31/407; A61K 31/497

(52) U.S. Cl. CPC A61K 35/50; A61K 9/08; A61K 9/0053; A61P 1/00; A61K 45/06; A61K 31/145; A61K 38/57; A61K 38/55; A61K 33/02; A61K 31/198; A61K 31/10; A61K 31/40; A61K 31/407; A61K 31/497

(57) ABSTRACT

A liquid formulation comprising an acellular extract of placental tissue and a solvent that may prevent the onset or reduce the symptoms of an intestinal condition in a subject when a therapeutically effective amount of the liquid formulation is delivered to a gastric system of the subject.

### CROSS-REFERENCE TO RELATED APPLICATION

This application claims the benefit of U.S. Provisional Application No. 62/941,382, filed Nov. 27, 2019, which is incorporated herein by reference in its entirety.

The Applicant/Assignee of this application has received U.S. government support under SBIR 1R44HD100243-01A1 awarded by the National Institutes of Health. The U.S. government may have certain rights in the invention.

### FIELD

The present disclosure relates generally to biological compositions and methods for their use. More specifically, but not by way of limitation, this disclosure relates to compositions derived from extracts of placental membrane and methods for their use.

### BACKGROUND

Intestinal disorders, including Necrotizing Enterocolitis (NEC), can impact quality of life and patient mortality. NEC and other intestinal disorders can lead to necrosis or perforation of the intestine and bowel that may require intestinal resection to remove diseased or damaged tissue. A perforated bowel can allow bacteria to move from the intestine into the abdominal cavity, and can lead to life-threatening infections, including peritonitis. Bowel resection may be required in many NEC cases. Most NEC cases occur in very premature infants that have a low birth weight. NEC lacks a cure and the mortality rate for neonates affected with NEC can be up to 30%. Therapies for NEC have included administration of broad-spectrum antibiotics, initiation of bowel rest, and the provision of fluid and inotropic support to maintain cardiorespiratory function. Other therapies target poor microbial defense of the premature gut and seek to normalize the gut microbiome with antibiotics and probiotics.

### SUMMARY

Described herein are formulations that may be used to prevent or treat intestinal conditions such as Necrotizing Enterocolitis (NEC) in a subject. For example, the formulations may be used prophylactically in a subject to prevent the occurrence of NEC. In some examples, the formulation may be a liquid that may be administered orally. Oral formulations may comprise an acellular extract of placental tissue and a solvent. In some examples, the placental tissue may comprise a placental disc. In some examples, the solvent may comprise milk, water, saline, a sodium lactate solution or a sugar solution. Optionally, the oral formulations may further comprise a diluent, an excipient, a carrier, or one or more exogenous protease inhibitors.

Also described herein are methods for preventing or reducing symptoms of an intestinal condition in a subject with a liquid formulation comprising an acellular extract of placental tissue and a solvent. In some examples, the methods may comprise obtaining a liquid. formulation and delivering a therapeutically effective amount of the liquid formulation to the gastric system of a subject. In some examples, the subject may be a premature infant having a birthweight of 1500 grams or less. In some examples, the liquid formulation may be delivered to gastric system orally by natural feeding or an orogastric tube or percutaneously by a gastric feeding tube or a duodenal feeding tube.

### BRIEF DESCRIPTION OF THE DRAWINGS

FIG. 1 is a chart of protein concentration amounts for placental disc extract with protease inhibitors, term amniotic fluid, and 16-20 week amniotic fluid.

FIG. 2 is a chart of jejunum cell growth for a range of placental disc extract protein concentrations.

FIG. 3 is a chart of jejunum cell growth for a range of placental disc extract protein concentrations.

FIG. 4 is a chart of MIF concentration for a range of placental disc extract protein concentrations in response to LPS stimulation.

FIG. 5 is a chart of IL-10 concentration for a range of placental disc extract protein concentrations in response to LPS stimulation.

FIG. 6 is a chart of TNF concentration for a range of placental disc extract protein concentrations in response to LPS stimulation.

### DETAILED DESCRIPTION

Certain aspects and features of the present disclosure relate to formulations comprising a placental tissue extract. In some examples, the formations may be used to prevent or treat intestinal conditions in a subject. For example, the formulations may be used prophylactically in a subject to prevent the occurrence of Necrotizing Enterocolitis (NEC).

NEC can be a life-threatening disease. NEC may affect neonates, with premature infants being most at risk for NEC. Most cases of NEC occur in very premature infants that have a birthweight less than 1500 grams (3.3 pounds). Immaturity of the intestine and its innate immune responses may be a factor in NEC occurrence in neonates. Other factors may be alteration in gut microbial colonization or hypoxia, ischemia, and/or reperfusion injury. In some cases, therapies for NEC may target the inflammatory propensity of the premature gut.

In some examples, an amniotic fluid substitute may hasten gut maturation and inhibit inflammation driven microvascular dysfunction that may initiate or exacerbate NEC. While in utero, a fetus can swallow amniotic fluid that may have anti-inflammatory properties and may prevent infection. During late gestation, a human fetus can swallow about 800 mL of amniotic fluid daily. Deprivation of amniotic fluid by premature delivery or premature rupture of membranes may delay maturation of the fetal gut or maturation may proceed more slowly.

In some examples, an amniotic fluid substitute may be dosed using the gastrointestinal tract. In some examples, the dosing may be performed orally. In some embodiments, an amniotic fluid substitute comprises: a cell-free preparation of full-term, post-delivery mammalian placenta for an acellular extract of placental tissue (described in U.S. patent application Ser. No. 16/321,075, filed Jan. 28, 2019, which is incorporated herein by reference), may closely resemble second-trimester amniotic fluid. The preparation can extract and preserve cytokines and growth factors stored within the placental disc and exclude amnion epithelial cells and the inflammatory chemokines that they synthesize and release into term amniotic fluid to initiate labor or to fight potential infection after rupture of membranes. The inflammatory chemokine milieu of an acellular extract of placental tissue may be lower than term amniotic fluid, especially in its IL-6 to IL-10 ratio. In some examples, a formulation comprising an acellular extract of placental tissue similar to 16- to 20-week amniotic fluid may prevent or reduce the occurrence of NEC in neonates. In some examples, a formulation comprising an acellular extract of placental tissue may reduce the severity of NEC in neonates.

Delivery of a formulation to a patient, especially a neonate, may be facilitated by using a liquid form that may be administered orally or percutaneously. In some examples, a formulation comprising an acellular extract of placental tissue may be in liquid form. In some examples, an acellular extract of placental tissue may be a shelf-stable, terminally sterilized crystalline powder. In some examples, a liquid formulation may comprise an acellular extract of placental tissue and a solvent. In some examples, a solvent may be used to dilute, dissolve, or suspend an acellular extract of placental tissue to form a liquid formulation. In some examples, the ratio of solvent to acellular extract in a liquid formulation for oral delivery may be different than the ratio for percutaneous delivery. The formulation may be administered during the transition between birth and normal feeding and may bolster and sustain gut maturation.

Disparate growth factor effectiveness together with success of vasodilatory therapies and broad chemokine milieu of amniotic fluid preparations may suggest multiple inciting etiologies exist. A therapeutic placental extract formulation may combine the ability to mature the fetal gut with factors that may be anti-inflammatory and vasodilatory. In some examples, hyperinflammatory milieu that can drive intestinal mucosal injury of the premature gut may be reduced.

### Oral Formulation

In some examples, a liquid formulation or oral formulation as described herein may comprise an acellular extract of placental tissue and a solvent. In some examples, the solvent may include milk, water, saline, Ringer's lactate solution, a dextrose solution, a sucrose solution, a synthetic sugar solution, or combinations thereof In some examples, the solvent may comprise natural mammalian milk or synthetic mammalian milk. In certain examples, the milk may be dehydrated and reconstituted with water, saline, Ringer's lactate solution, a dextrose solution, a sucrose solution, a synthetic sugar solution, or combinations thereof.

In some examples, the acellular extract may be from placental tissue that comprises a placental disc. In some examples, the acellular extract may be from placental tissue that comprises a villous chorion. In certain examples, the acellular extract may be from placental tissue that comprises a placental disc and a villous chorion.

In some examples, the concentration in the liquid formulation of one or more proteins from the acellular extract may be up to 25 mg/mL (e.g., 2-10 mg/mL, 4-15 mg/mL, 3-20 mg/mL). For example, the concentration of one or more proteins may be 0.01 mg/mL, 0.02 mg/mL, 0.05 mg/mL, 0.1 mg/mL, 0.15 mg/mL, 0.2 mg/mL, 0.25 mg/mL, 0.5 mg/mL, 0.75 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or up to 25 mg/mL.

In some examples, the oral formulation may further comprise a diluent, an excipient, a carrier, or combinations thereof. Optionally, the oral formulation may further comprise one or more exogenous protease inhibitors. For example, the oral formulation may comprise one or more of 4-(2-Aminoethyl) benzenesulfonyl fluoride hydrochloride (AEBSF), Bovine Lung Aprotinin, N-(trans-Epoxysuccinyl)-L-leucine 4- guanidinobutylamide, Leupeptin, N-ethylmaleimide (NEM), phenylmethylsulfonylfluoride (PMSF), ethylenediamine tetraacetic acid (EDTA), ethylene glycol-bis-(2-aminoethyl(ether)NNN′N′-tetraacetic acid, ammonium chloride, boceprevir, danoprevir, narlaprevir, telaprevir, or vaniprevir. In some examples, the oral formulation may further comprise vitamins or minerals. In certain examples, the oral formulation may further comprise an antibiotic.

### Methods of Using Placental Extract Formulations

Also described herein are methods for preventing or reducing symptoms of an intestinal condition in a subject. The methods may comprise obtaining a liquid formulation; and delivering a therapeutically effective amount of the liquid formulation to a gastric system of the subject. The liquid formulation may comprise an acellular extract of placental tissue and a solvent. In some examples, the intestinal condition may comprise Necrotizing Enterocolitis (NEC).

In some examples, the subject may be a human. For example, the subject may be a newborn infant. In. certain examples, the subject may be an infant born prematurely, or before 37 weeks of gestation. In some cases, an infant, or neonate, may have a low birthweight. For example, a premature infant may have a birthweight of 1500 g or less. In some examples, the subject may be a mammal. For example, the subject may be an equine, a bovine, an avis, a capra, a porcine, a canine, a feline, a ursidae, a primate, a camelid, or other.

In some examples, a subject may be less than 100 days of age (e.g., less than 90 days, less than 45 days, less than 21 days, less than 10 days, less than 3 days, less than 1 day). For example, the age of a subject may be less than 100 days, 95 days, 90 days, 85 days, 80 days, 75 days, 70 days, 65 days, 60 days, 55 days, 50 days, 45 days, 40 days, 35 days, 30 days, 29 days, 28 days, 27 days, 26 days, 25 days, 24 days, 23 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or less than 24 hours.

In some examples, the liquid formulation may be administered prophylactically to prevent the onset of an intestinal condition. In other examples, the liquid formulation may be administered to reduce the severity of symptoms or duration of an intestinal condition. In some examples, the liquid formulation may be delivered to the subject orally by natural feeding or an orogastric tube. The timing of delivery may be coordinated with the timing of meals and delivered to the subject contemporaneous with periodic feedings of the subject. In some examples, the liquid formulation may be provided with a nutrient feeding. In some examples, the liquid formation may comprise natural mammalian milk, synthetic mammalian milk, water, saline, a sodium lactate solution, or a sugar solution.

In other examples, the liquid formulation may be delivered to the subject percutaneously through a feeding tube. For example, a gastric feeding tube or a duodenal feeding tube may be used to deliver the liquid formulation. In some examples, the liquid formulation may be provided with other pharmaceutical or therapeutic treatments.

In some examples, the liquid formation may be administered to the subject in a continuous manner or at a frequency of 1 hour, 2 hours, 3 hours, 4 hours, 5 hours, 6 hours, 7 hours, 8 hours, 9 hours, 10 hours. 11 hours, 12 hours, 18 hours, 24 hours, 36 hours, 48 hours, or 72 hours. In some examples, the liquid formation may be administered to the subject for a treatment period up to 100 days (e.g., up to 90 days, up to 45 days, up to 21 days, up to 13 days, or up to 5 days). For example, the liquid formation may be administered to the subject for a treatment period up to 100 days, 95 days, 90 days, 85 days, 80 days, 75 days, 70 days, 65 days, 60 days, 55 days, 50 days, 45 days, 40 days, 35 days 30 days, 20 days, 18 days, 16 days, 14 days, 12 days, 10 days, 8 days, 7 days, 6 days, 5 days, 4 days, 3 days, 2 days, or 1 day,

In some examples, a therapeutic amount of the liquid formulation may prevent the onset of an intestinal condition such as NEC. In some examples, a therapeutic amount of the liquid formulation may reduce severity or symptoms of an intestinal condition. For example, a therapeutic amount of the liquid formulation may reduce inflammation on in the intestine of the subject. In some examples, a villous height of the intestine of the subject may be increased. In some examples, a crypt depth of Me intestine of the subject is increased.

### Intestinal Condition NEC

In some examples, a liquid formulation may be administered to a subject with intestinal conditions comprising NEC. The inflammatory response to NEC can lead to mucosal barrier breakdown, bacterial translocation, and loss of GI structural integrity. A formulation comprising an acellular extract of placental tissue may decrease the inflammatory response, and may increase the proliferative capacity of intestinal stem cells to enable the intestine or bowel to return to its original function. In some examples, local dosing of high concentrations of the anti-inflammatory and immunomodulatory chemokines in formulation comprising an acellular placental extract could also be a treatment strategy for NEC.

### Amniotic Fluid

Maternal milk, especially colostrum, can stimulate normal gastrointestinal development and maturation, and can regulate an immature gut immune system. Colostrum may include antimicrobial proteins (i.e. immunoglobulins, casein, lysozyme, lactoferrin) and anti-inflammatory cytokines and growth factors, such as the anti-inflammatory cytokine interleukin (IL)-10, transforming growth factor (TGF), and epidermal growth factor (EGF). Amniotic fluid may include many of the factors found in maternal milk.

There are currently no treatments that mimic amniotic fluid or its function in the fetal gut for neonates. Animal models for NEC have shown a decrease in the incidence of when feedings are supplemented with amniotic fluid. Not intending to be bound by theory, anti-inflammatory cytokines and growth factors, including epidermal growth factor, hepatocyte growth factor, and IL-10, in amniotic fluid may be effective at preventing NEC. Amniotic fluid, whose protein constituency is supplied by the placenta, may contain high concentrations of immunomodulatory peptides such as IL-10 and may contain growth factors such as EGF, insulin-like growth factor 1 IGF-1, and TGF, as shown in Table 1. Reduced fetal intestinal growth without exposure to amniotic fluid may suggest the constituents of amniotic fluid may be important to gastrointestinal development in utero and may help suppress inflammatory responses. At birth, a premature infant loses the immunomodulatory and developmental benefits of swallowing amniotic fluid.

TABLE 1

| Trophic factor | Reported role in intestinal growth |
|---|---|
| Epidermal Growth Factor | Intestinal cell proliferation and survival. |
| (EGF) | |
| Hepatocyte Growth Factor | Intestinal cell proliferation and injures |
| (HGF) | intestinal tissue repair. |
| Transforming Growth Factor | Intestinal mucosal repair. |
| (TGF-α and TGF-β) | |
| Insulin-like Growth Factor 1 | Intestinal cell proliferation and growth. |
| (IGF-1) | |
| Erythropoietin (EPO) | Increases villus height, villus area, crypt |
| | depth, and crypt epithelial cell |
| | proliferation. |
| Granulocyte Colony- | Epithelial cell maintenance and protection. |
| Stimulating Factor (G-CSF) | |
| Interleukin (IL) family | Enhances intestinal epithelial cell |
| | restitution. Intestinal epithelial cell |
| | proliferation and increased nutrient uptake. |

While in utero, swallowed amniotic fluid can bathe immature enterocytes in placenta-derived immunoregulatory, antimicrobial, and growth-promoting factors. The factors may prepare the gut for a shift from a highly-controlled in utero environment to the exceptionally stressful environment immediately after birth. In a fetal sheep model, interruption of amniotic fluid ingestion by esophageal ligation was shown to cause mucosal atrophy, villus blunting, and enterocyte abnormalities. In that model, effects of interrupted amniotic fluid swallowing on the intestinal mucosa were gradually reversed after esophageal ligatures were removed and amniotic fluid ingestion resumed. Effects were not reversed by infusion of lactated Ringer's solution alone.

Amniotic fluid has shown decreased NEC severity in a 10-day-old hypoxic mouse model of NEC via Toll-like receptor (TLR) 4 and EGR receptor pathways. Postnatal administration of porcine amniotic fluid as minimal enteral nutrition to preterm piglets has shown increased body weight gain, altered bacterial colonization and NEC severity, and induced differential expression of mRNA coding for genes involved in gut inflammatory responses.

Intra-peritoneal administration of amniotic fluid stem cells has shown improved survival and enhanced repair in a rat model of NEC. In that study, intraperitoneal administration of amniotic fluid stem cells was associated with the migration of cyclooxygenase 2 (COX-2)-expressing stromal cells from the lamina propria of the small intestinal villi to a position near the base of the intestinal crypts. The beneficial effects of amniotic fluid stem cells on NEC were blocked by a selective COX-2 inhibitor, suggesting that migration of these COX-2—expressing stromal cells was involved in protective effects of amniotic fluid stem cells. Clinical scalability is currently not feasible as the median volume of amniotic fluid obtained at cesarean section is approximately 70 mL.

### EXAMPLES

### Example 1

An acellular placental extract was prepared as follows. Placenta/amniotic membrane was procured from a contract hospital via a placenta donation, and the human placental homogenate was isolated. The placenta/amniotic membrane was placed in a 0.9% normal saline solution (NS, 154 mM NaCl, 308 mOsm/L). The placenta was removed from the 0.9% NS, the placental disc was separated from the amniotic membrane, chorion, and umbilical cord, and dissected into pieces approximately 1.5 inches by 2 inches. These smaller pieces of placental disc were placed in a container with approximately 400 mL of 1×phosphate buffered saline (PBS, 137 mM NaCl, 2.7 mM KCl, 4.3 mM Na2HPO₄, 1.47 mM KH₂PO₄, pH 7.4). The container was placed on a shaking platform and shaken at 120 rpm for 12 to 24 hrs. The placental disc pieces were grossly homogenized using a laboratory blender with 1×PBS or 1×PBS that contained protease inhibitor (PI) at a 1×final concentration (AEBSF 500 uM, Aprotinin 150 nM, E-64 1 uM, Leupeptin 1 uM). The gross homogenate was placed into 250 mL containers and centrifuged for 10 minutes at 4,000 times gravity. The gross homogenate was poured into the container for a ratio of approximately 1:5, tissue volume: total volume. The container was vortexed/shaken to re-suspend the pellet. The suspension was centrifuged for 10 minutes at 4,000 times gravity. This wash procedure was repeated for a total of three washes to remove blood from the tissue. The mixture was not centrifuged after the last wash. The contents of the 250 mL containers were diluted with either 1×PBS or I×PBS-1×P1 to double the volume. The mixture was then placed into a reservoir feed connected to a high-pressure homogenizer. The homogenate was then placed into 250 mL containers and centrifuged at 15,000 times gravity for 10 minutes.

A Bio-Plex MAGPIX Multiplex Reader (Bio-Rad Laboratories, Hercules, Calif.) was used to quantify the amounts of cytokines and growth factors present in the solution extracted from the placental disc. A Bio-Plex Pro Human Chemokine plane, 40-plex assay (Bio-Rad Laboratories, Hercules, Calif.) was obtained. The standards were diluted in appropriate diluent. Assay beads were diluted and 50 μL was added to each well of the 96-well plate and washed twice with assay buffer. The plate was placed on a magnetic plate holder and the solution removed from the wells. The standards, samples, and blanks were loaded into each respective well of the 96-well plate and incubated for 1 hour at room temperature (duplicate measurements were performed on each standard, sample and blank). The plate was washed by placing the plate on the magnetic plate holder, removing the solution, washing with wash buffer, and removing the wash buffer by placing the well plate on the magnetic plate holder. The detection antibodies were added to each well and incubated for 30 minutes at room temperature. The plate was washed 3 times with wash buffer and the streptavidin-PE indicator was added to each well. The plate was incubated for 10 minutes in the dark at room temperature. The plate was washed 3 times in assay buffer and measurements were taken on the MAGPIX multiplex reader. Data was analyzed on Bio-Plex reader software (Bio-Rad Laboratories, Hercules, Calif.) and a two-way ANOVA was utilized to compare the treatment groups.

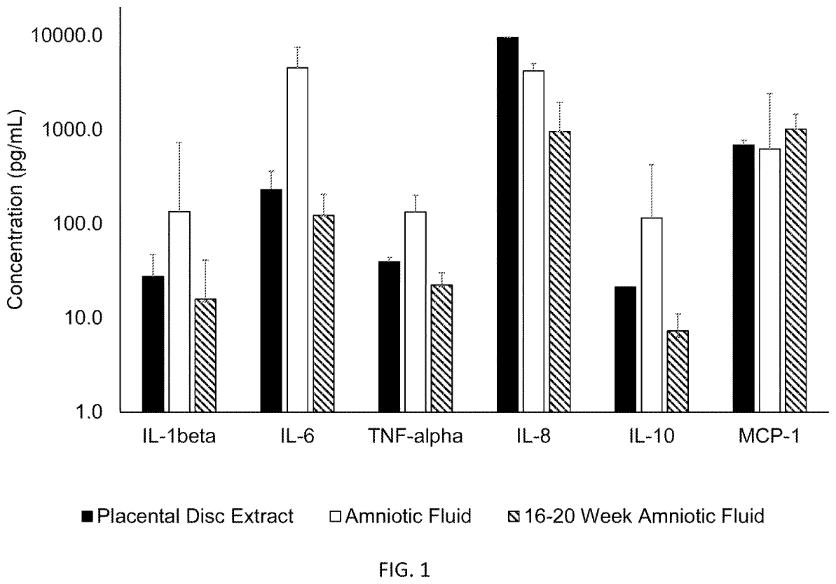

The chemokine profile of the acellular placental extract was similar to the published chemokine profiles of 16- to 20-week amniotic fluid. The yield of the acellular placental extract was about two liters per placenta before lyophilization. A comparison of protein concentrations present in term amniotic fluid (n=10), acellular placental extract (n=10, donor matched with amniotic fluid), and 16-20-week amniotic fluid (from published results) is shown in FIG. 1. Concentrations of term amniotic fluid and the acellular placental extract were measured using a Bio-Plex Pro Human Chemokine panel. The values for 16-20 amniotic fluid were as reported in literature. As shown in Table 2, the inflammatory components in the acellular placental extract were significantly lower than those in term amniotic fluid. A 33% reduction in % coefficient of variation was observed in the acellular placental extract as compared to term amniotic fluid, using a paired t-test with a significance of p<0.05.

TABLE 2

| | Term amniotic | Acellular placental |
|---|---|---|
| | fluid | extract |
| Availability | Cesarean section | FDA-approved issue bank |
| Yield | 70 mL/delivery | 2000 mL/placenta |
| Coefficient of variation | 98% | 69% |

Inflammatory Cytokines

| | | |
|---|---|---|
| TNF-α, pg/mL | 133 | 40 |
| IL-1, pg/mL | 135 | 28 |
| IL-6, pg/mL | 4534 | 230 |

Anti-Inflammatory Cytokines

| | | |
|---|---|---|
| IL-10, pg/mL | 115 | 97 |
| IL-8, pg/mL | 4194 | 43405 |
| MIF, pg/mL | 241314 | 702283 |

Growth-Related Cytokines

| | | |
|---|---|---|
| GRO- α, pg/mL | 4081 | 4851 |
| SDF-1, pg/mL | 1149 | 1226 |
| TEC, pg/mL | 2916 | 4608 |

Tumor Necrosis Factor-alpha (TNF-α),
Macrophage migration inhibitory factor (MIF),
Growth-regulated oncogene-alpha (Gro-α),
Stromal cell-derived factor (SDF-1),
Thymus Expressed Chemokine (TEC)

A characterization of the acellular placental extract was conducted using a Bioplex Multiplex assay. Table 3 shows the proteins that were identified the extract. Many may have vital functions in the gut maturation process.

TABLE 3
(n = 24)

| Chemokines | | | Cytokines | |
|---|---|---|---|---|
| 6Ckine/CCL21 | GRO-β/CXCL2 | MIG/CXCL9 | | |
| BCA-1/CXCL13 | I-309/CCL1 | MIP-1α/CCL3 | IL-10 | IL-6 |
| CTACK/CCL27 | I-TAC/CXCL11 | MIP-1δ/CCL15 | IL-16 | MIF |
| ENA-78/CXCL5 | IL-8/CXCL8 | MIP-3α/CCL20 | IL-1β | TNF- α |
| Eotaxin/CCL11 | IP-10/CXCL10 | MIP-3β/CCL19 | IL-2 | IL-1ra |
| Eotaxin-2/CCL24 | MCP-1/CCL2 | MPIF-1/CCL23 | IL-4 | |
| Eotaxin-3/CCL26 | MCP-2/CCL8 | SCYB16/CXCL16 | | |
| Fractalkine/CX3CL1 | MCP-3/CCL7 | SDF-1/CXCL12 | Growth Factors | |
| GCP-2/CXCL6 | MCP-4/CCL13 | TARC/CCL17 | PDGF-β | bFGF |
| GRO-α/CXCL1 | MDC/CCL22 | TEC/CCL25 | | EGF |

### Example 2

An acellular placental extract was prepared using the method of Example 1 until the step of high pressure homogenization. In place of high pressure homogenization, the mixture was placed into freezer safe containers and frozen at −80° C. Once frozen, the material was removed from the freezer, thawed in a 40° C. water bath, and placed into centrifuge containers. The material was sonicated for 5 minutes at 400 W by placing the sonicator tip in the solution for one minute, then removing for at least one minute to cycle the process. The lysed cell solution was centrifuged at 15,000 times gravity for 30 minutes.

A determination of whether intestinal epithelial cells (jejunum cells) proliferated or differentiated in response to increasing concentrations of the acellular placental extract was conducted. To measure proliferation, the total number of cells in a 1,600×1,600-pixel square was determined. To measure cell differentiation, the expression of an intestinal stem cell marker (leucine-rich-repeat-containing G-protein-coupled receptor 5 [Lgr5]), a goblet cell marker (Mucin 2 [Muc2]), an enterocyte marker (Sucrase-Isomaltase [SI]), and a Paneth cell marker (Lysozyme [Lyz]) were evaluated.

Experimental Design: Jejunum cells were grown for 1 day in 25% Altis Expansion medium in a 37° C. tissue culture incubator with 5% CO₂. Twenty-four hours post-plating, the medium was replaced with a medium containing increasing concentrations of acellular placental extract. Cells were grown for 48 hours and then collected for final time points outlined in experiments below.

Proliferation study: Cultures were then fixed with 4% paraformaldehyde for 20 minutes at room temperature. Immunostaining for visualization of CD-326 (EPCAM) protein, and nuclei (Hoescht stain) was performed. Wells were individually tile scanned using a fluorescent scope and automated stage at 4× for each fluorescent wavelength of interest. Images were stitched together using Image J software and overlaid to visualize co-localization of immunostaining. A 1,600×1,600-pixel square was isolated from the middle of each well and was quantified for: total number of cells (Hoechst+; EPCAM+). To measure confluence, Image J software was used to threshold the entire well image of EPCAM immunostaining and create a binary mask. The mask was then enhanced by dilating each pixel and the area of pixels was measured with the total area of each well.

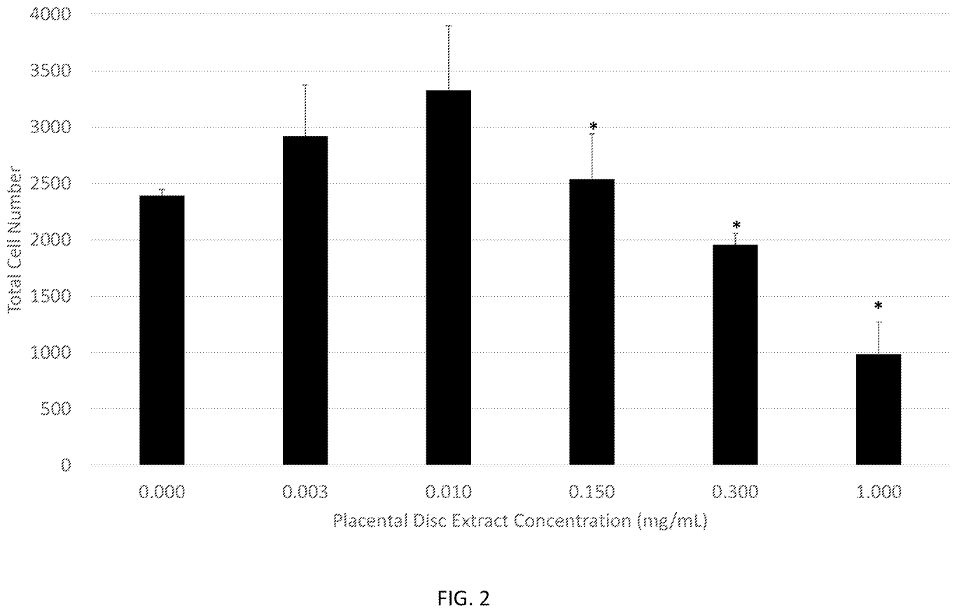

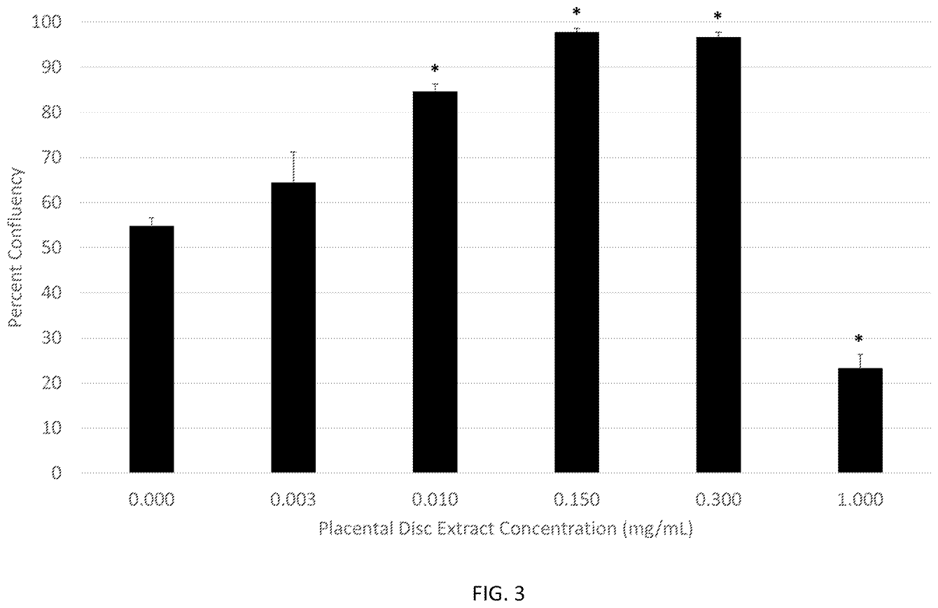

Results: As shown in FIG. 2, the total number of cells increased with 0.003-0.01 mg/mL of acellular placental extract protein, but decreased with concentrations of acellular placental extract protein greater than 0.15 mg/ml. While the apical side of the intestine may be exposed to approximately 4 mg/mL total protein from the amniotic fluid in utero, the concentration presented to the stem cells at the bottom of the crypt can be about 1/100^{th} of the levels seen on the top. Confluency measurements also showed that the confluency of cells per well was significantly increased with increased concentrations of acellular placental extract protein (between 0.003 and 0.3 mg/mL), and then decreased as acellular placental extract protein concentration reached 1 mg/mL (see FIG. 3). FIGS. 2 and 3 show jejunum cell growth in response to acellular placental extract. FIG. 2 shows total cell number increased with acellular placental extract concentrations between 0.003 and 0.01 mg/mL, and then decreased with protein concentrations greater than 0.15 mg/mL. FIG. 3 shows the percent coverage of each well significantly increased with doses of 0.01-0.3 mg/mL and then decreased in the 1 mg/mL dose of acellular placental extract. A one-way ANOVA with Tukey post-hoc test compared to the 0 mg/mL acellular placental extract treated wells had a significance of p<0.01. Bars are standard deviation of n=3.

Differentiation study results: Gene expression analysis of cell differentiation is shown in Table 4. Intestinal stem cells and goblet cells increased in the 0.01 and 0.15 mg/mL acellular placental extract treatments. Enterocytes increased in the 0.003-0.3 mg/mL treatments. An increase in Paneth cells at the 0.003-0.15 mg/mL acellular placental extract treatments was observed.

TABLE 4

| | | Acellular placental extract concentration | | | | |
|---|---|---|---|---|---|---|
| | | (mg/ml) | | | | |
| Cell type | Control | 0.003 | 0.01 | 0.15 | 0.3 | 1 |
| Intestinal stem cell | + | ++ | +++ | ++ | ++ | +++ |
| Goblet cell | + | ++ | ++ | ++ | ++ | +++ |
| Paneth cell | + | ++ | ++ | + | + | ++ |
| Enterocyte | + | +++ | +++ | +++ | ++ | + |

### Example 3

The acellular placental extract from Example 2 was used for a Transwell study. A cell monolayer platform was used to specifically measure changes to the epithelial barrier function. This system allowed the measurement of both transepithelial resistance and flux across the epithelial barrier.

Experimental design: Cells were passaged into 48 transwells on 4 plates and grown in growth medium for 5 days. On day 6, medium was changed to a differentiation medium and grown for 4 days. On day 9 treatments with increasing dose of the acellular placental extract (0, 0.25, 1, and 4 mg/ml) were added to the apical side along with increasing doses of LPS (0, 100, 250, and 500 ng/ml) to stimulate an inflammatory response within the cells. Medium on the basolateral side was collected 48 hours post treatment and measured for IL-1, IL-6, TNF-α, and MIF by ELISA per manufacturer instructions (R&D Systems, and Thermofisher Scientific). Interactions between treatments were analyzed with two-way ANOVAs.

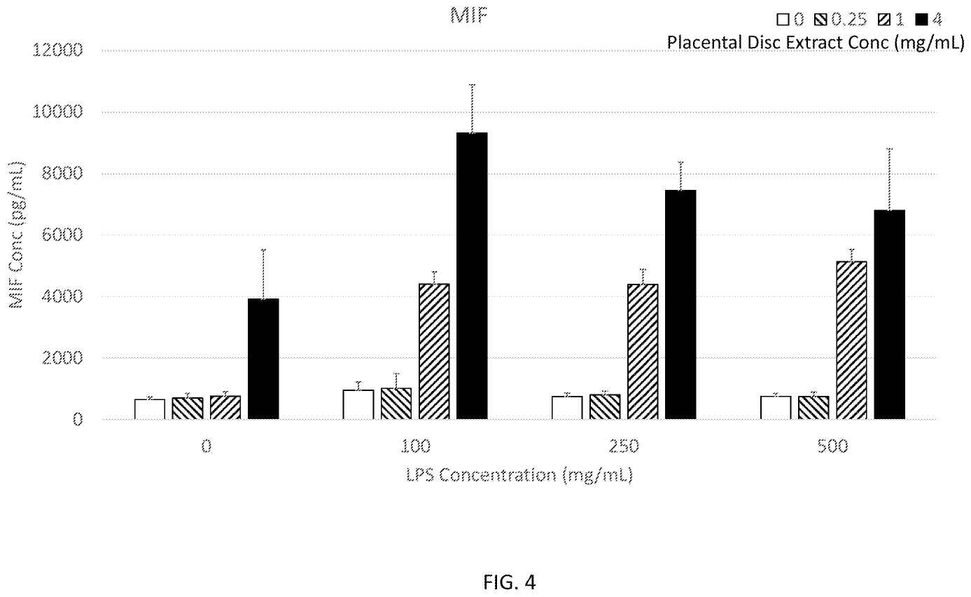

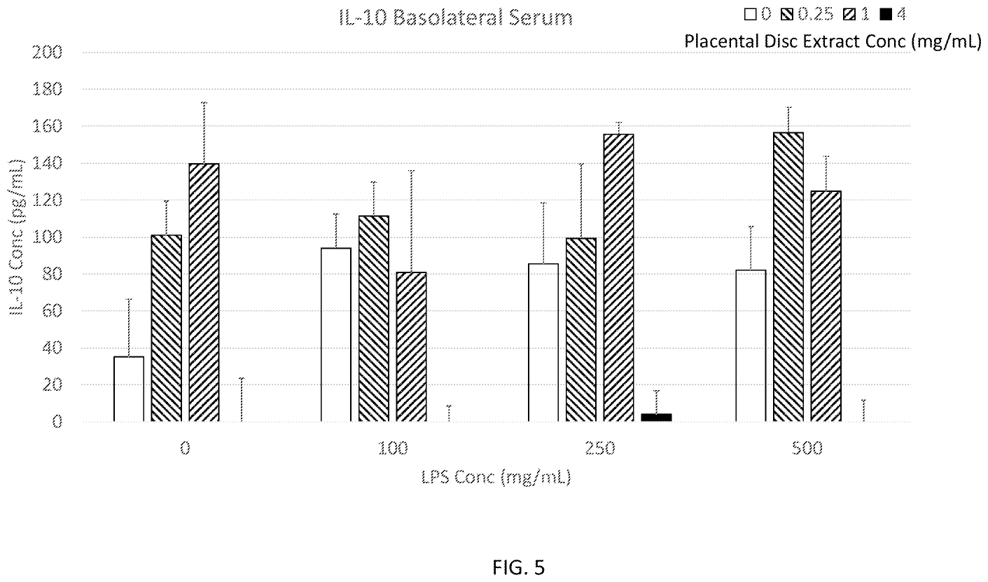

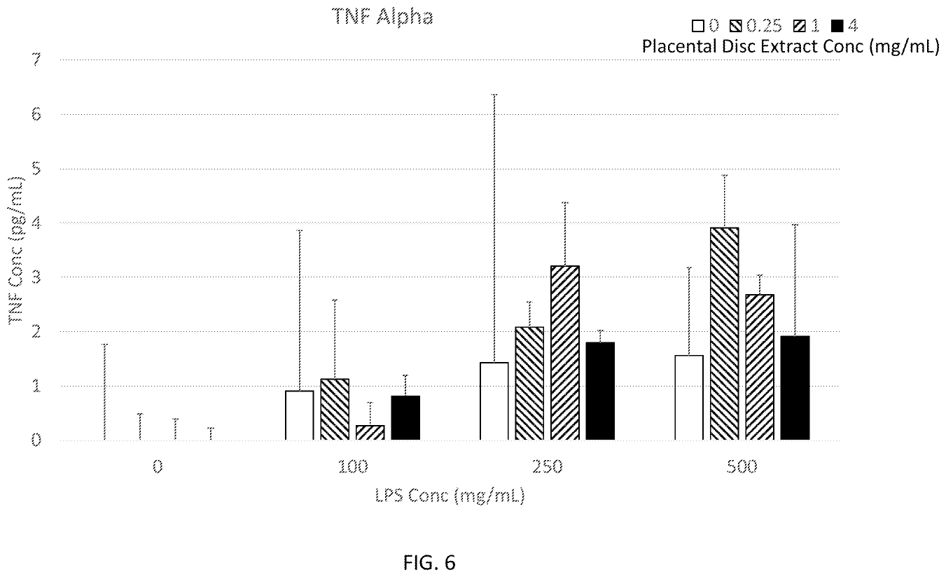

Results: As shown in FIGS. 4-6, the acellular placental extract had beneficial effects on the jejunum cells not exposed to LPS. FIGS. 4-6 shows jejunum cell protein secretion in response to LPS Stimulation. Cells were grown in single layers in transwell plates. Treatments of LPS (0, 100, 250, and 500 ng/mL) and acellular placental extract (protein levels of 0, 0.25, 1, and 4 mg/mL) were added to the apical side of the transwell, each treatment was performed in triplicate. The medium from the basolateral side of the transwell was measured for MIF, and TNF-α by ELISA. All proteins showed a statistically significant interaction (p<0.01) when performing a two-way ANOVA.

TNF-α was unaltered at any dose given, MIF increased at the 4 mg/mL dose while other doses had no effect, and IL-10 increased in the 0.25 and 1 mg/mL doses and was not observed in the 4 mg/mL dose. In response to LPS, the acellular placental extract continued to decrease inflammatory responses and increase anti-inflammatory responses. The results from a two-way ANOVA on each protein reveal a statically significant interaction between the LPS and acellular placental extract treatments (MIF p<0.0001, TNF-α p<0.01, IL-10 p<0.01).

These in vitro results demonstrate the acellular placental extract can increase cell number, coverage, and differentiation of several intestinal cells. acellular placental extract can also counteract inflammatory responses by increasing anti-inflammatory proteins secreted from the basolateral side of the cells in culture and decreasing the inflammatory proteins in the same assay.

### Example 4

An in vivo study of acellular placental extract was conducted. A widely used piglet model of NEC was used to test the efficacy of the acellular placental extract. An acellular placental extract was prepared using the method of Example 2, except eight placentas were processed. Centrifuged liquid from all 8 placentas was pooled into a single container and thoroughly mixed. The total protein of the solution was measured via a Bradford protein assay (ThermoScientific). The solutions were aliquoted into 50 mL containers such that each container contained 120 mg of total protein. The 50 mL containers were lyophiled (freeze dried), and then sterilized with 2 MRad of gamma irradiation.

Experimental design: The piglets used were delivered on day 105 of gestation (115 days term), equivalent to a 32-week human pregnancy, according to the protocol established by Bjornvad et al. Immediately following delivery, piglets (n=9) received acellular placental extract via an oral gastric tube for the first 48 hours of life. A control group (n=3) of “standard of care” (SOC) (IV feeds only, just as premature babies receive in the intensive care nursery). Forty-eight hours after delivery, acellular placental extract treatment and IV feedings were discontinued, and piglets were transferred to oral formula. Animals were sacrificed per IACUC guidelines 48 hours after the initiation of oral formula feeds. At sacrifice, necropsies were performed, and the small intestine were divided into thirds. A 5-10 cm piece from each section was fixed, embedded with paraffin, sectioned, and stained with hemotoxylin and eosin (H&E). The intestines, liver, lungs, heart, and kidneys were preserved in Formalin for subsequent histopathological analysis.

Survival and necropsy analysis: The 48-hour survival rate of the acellular placental extract group was twice that of SOC (6/9 acellular placental extract vs. ⅓ for SOC). Historical number of control piglets is shown in Table 5. Compared to the 42 −77% incidence of clinical NEC with SOC, acellular placental extract eliminated the clinical incidence of NEC. At necropsy, the gastrointestinal tract was removed and analyzed.

TABLE 5

| Number of piglets that | Total number of | |
|---|---|---|
| developed NEC | piglets | Source |
| 5 | 12 | Bjornvad et al. 2005 |
| 137 | 283 | Bjornvad et al. 2008 |
| 17 | 22 | Siggers et al. 2008 |
| 26 | 46 | Sanglid et al. 2006 |
| 13 | 22 | Thymann et al. 2009 |
| 28 | 40 | Ghoneim et al. 2014 |
| 21 | 38 | Zamora et al. 2015 |
| 0 | 9 | Acellular placental extract |
| | | treated piglets |

To characterize the extent of NEC damage, the following clinical NEC scoring system was applied as indicated by macroscopic evidence of inflammation, edema, hemorrhage, and necrosis, in the small intestine: (1) no or minimal focal hyperemic gastroenterocolitis; (2) mild focal gastroenterocolitis; (3) moderate locally extensive gastroenterocolitis; (4) severe locally extensive hemorrhagic gastroenterocolitis; (5) severe locally extensive hemorrhagic and necrotic gastro enterocolitis; and (6) severe diffuse hemorrhagic and necrotic gastroenterocolitis. Piglets given a NEC score of 3 or higher for one gastrointestinal section were considered positive for NEC. The intestines of piglets treated with acellular placental extract showed no clinical signs of NEC at necropsy: 0/9 piglets scored ≥3.

Histopathologic analysis: H&E stained sections were blindly examined by a board-certified pathologist. Histopathologic analysis of small intestine is shown in Table 6. The acellular placental extract treated group showed no histological signs of NEC while ⅓ SOC animals presented with histological NEC. Villous height and crypt depth were measured in 10 villi per section. Acellular placental extract significantly increased villous height and crypt depth (p<0.0001) compared to the standard of care control group.

TABLE 6

| | Villous height (μm) | | Crypt dept (μm) | |
|---|---|---|---|---|
| | AVG | STE | AVG | STE |
| Control (SOC) | 371.7 | 104.4 | 76.8 | 21.7 |
| acellular placental extract | 465.6 | 58.0 | 88.0 | 7.5 |

### Example 5

Specific Aim 1. To develop an in vitro bioactivity and potency assay of acellular placental extract to predict in vivo performance.

Rationale: acellular placental extract may (1) increase the proliferation of intestinal cells in culture; (2) demonstrate that acellular placental extract may beneficially alter the cellular response to hypoxia, a stress seen in vivo as the vasculature of the intestine is under-developed and poor blood/gas/nutrient exchange lead to increased permeability of the intestinal epithelium. The porcine cell culture experiment proposed here may mimic the animal model proposed in Specific Aim 2.

Experimental Design: Tissue will be taken from 1- to 3-day-old male and female wild-type Yorkshire piglets euthanized for study research purposes. An 8-10 cm segment of the ileum will be excised and opened longitudinally. The tissue will be incubated in PBS containing EDTA, Y-27632, DTT, and penicillin/streptomycin at 4° C. Tissue will be transferred into a pre-warmed solution of PBS, EDTA, Y27632, and penicillin/ streptomycin, incubated at 37° C. and shaken to mobilize the crypt/villi units. The remnant intestine will be removed from the solution and the crypt component will be enriched, quantified, and pelleted. These crypts will be used to grow monolayers or frozen for future experiments to determine the capabilities of acellular placental extract to enhance proliferation, TEER, and permeability, and then determine the effects of acellular placental extract on hypoxia-induced stress.

Cell Proliferation: Cells isolated from the procedure described above will be cultured in 96-well plates. Treatments of acellular placental extract will be determined by total protein with the highest dose utilized being that of term amniotic fluid (4 mg/mL). From preliminary experiments, this dose is determined to be on the toxic side of the dose response curve, but still clinically relevant due to the concentration gradient observed within the crypts in vivo. Serial dilutions will be performed on the high dose of 4 mg/mL protein to achieve a lowest dose of 0.002 mg/mL protein. This lowest dose corresponds to the lowest dose tested in the preliminary work that is not significantly different than the untreated controls. Monolayer island size will be measured as well as the time to achieve 100% confluence as an indication of proliferation.

TEER evaluation and permeability: Cells isolated from procedure described above and plated in transwell plates. Monolayers will be tested for intestinal integrity and permeability with a dual planar electrode instrument (Epithelial Volt/Ohm Meter [EVOM]) following the manufacturer's instructions. Cells will be exposed to a hypoxic environment (1% O₂, 5% CO₂, and balanced with N₂ and humidified) or a normoxic environment (21% 02, 5% CO₂, and balanced with N₂ and humidified) as described previously. TEER values will be monitored daily for a total of 10-11 days during monolayer development. Data will be expressed as resistance per square centimeter. Permeability will be determined by placing Fluorescein isothiocyanate-dextran (FD4) on the apical side of the transwell 24 hours before the end of the experiment and quantifying the amount in the basolateral compartment. The doses of acellular placental extract for assessment of permeability will be determined by the most effective concentrations of acellular placental extract in increasing cellular proliferation from the above experiment.

Quantification of cellular response to hypoxia: Medium from the basolateral side of each transwell will be collected and frozen at the termination of each experiment. HIF-1 will be quantified to determine if any alterations are observed in response to hypoxia. ELISAs for IL-10, TNF-α, IL-6 (R&D Systems) will be carried out per the manufacturer's instructions to examine any modification to the inflammatory response to the hypoxia model. Finally, signs of a trophic response to hypoxia will be monitored by the presence of VEGF, EGF, and IGF-1.

Statistical Analyses: All data from this aim is quantitative data and will be analyzed via a two-way ANOVA with a Tukey post hoc testing to determine significance between specific groups.

Expected Results, Potential Problems, and Alternative Approaches: Acellular placental extract increased the number of human intestinal stem cells in previous studies. The study seeks to demonstrate whether the same effects occur in porcine ileum intestinal stem cells. No issues are anticipate any with these assays as there are published results showing that the team and amniotic fluid can positively identify and affect porcine intestinal epithelial cells. The down-selection of doses from the proliferation studies to the TEER and permeability assay introduces the possibility of missing the optimal dose to demonstrate the effect can be observed. Should no effect be observed, a broadening of the doses used for the TEER and permeability assays will be undertaken. In preliminary data, acellular placental extract increased cell numbers in response to LPS and decreased incidence in NEC observed in the pre-mature piglet model. These results increase the likelihood of success in these in vitro assays.

Results may include: (1) A dose response curve demonstrating the effect acellular placental extract on proliferation of porcine intestinal cells. (2) The ability of acellular placental extract to maintain TEER and decrease permeability in response to hypoxic stress. (3) The cellular response to hypoxic stress is altered when treated with acellular placental extract.

Specific Aim 2. To evaluate the efficacy and dose response of acellular placental extract to prevent NEC in premature piglets.

Rationale: A premature piglet NEC model will be used for this study. The model utilizes piglets delivered between 102 and 105 days gestation and has been described extensively. The key attributes of this model include similar size to human infants (0.6 to 1.1 kg), and impaired respiratory, nutritional, immunological, and metabolic responses. Preterm piglets are also tolerant of the handling procedures and have gastrointestinal characteristics similar to human infants. This model is used to generate preclinical data regarding breast milk and probiotic products now being tested in clinical trials. Acellular placental extract will be administered from delivery until enteral feedings commence 48 hours post-delivery, as described in the preliminary data. This will suffice to replace the amniotic fluid components clearly missing when the infant is delivered early. Acellular placental extract will be administered at a dose rate comparable to that of an infant swallowing amniotic fluid in utero (therapeutic dose, 4 mg/mL total protein at 2.5 mL/kg/h) and at 2 escalating doses to test whether increasing the amount of acellular placental extract presented to the intestinal wall has beneficial effects.

Experimental Design: Timed pregnant Yorkshire sows that have had at least 12 piglets in their first litter will be received at the research facility at approximately 95 days of gestation. The second-litter sows will be used to ensure that sufficient piglets are delivered by 6 sows, as second-parity sows have an increased litter size. Piglets will be delivered via C-section on day 102-105 gestation, randomly assigned to a treatment group in a sampling scheme of 1 standard-of-care to 3 of acellular placenta extract, and treatment administered within 3 hours of birth. Acellular placental extract treatment will be administered via an oral gastric tube at a rate of 2.5 mL/kg/h for the first 24 hours of life, and 5 mL/kg/h for the second 24 hours of life. The acellular placental extract will be administered at a total protein concentration similar to amniotic fluid (4 mg/mL, n=33). Control animals (n=11) will not be given any therapy into the intestine, as is customary in human premature babies. The escalating-dose studies will begin after the efficacy studies are completed. Again, piglets will be randomly assigned to a group after birth and before initiating treatments. Treatments of 2×(8 mg/mL) and 5×(20 mg/mL) acellular placental extract are planned to evaluate the effect of increased doses on the system. All animals will be given total parenteral nutrition (TPN) via an umbilical catheter for the first 48 hours; then TPN will be stopped and formula feedings initiated, as previously described. Piglets will be sacrificed 48 hours after initiating formula feedings and necropsy performed. The primary outcome of the study will be clinical observation of NEC in the piglets. Secondary outcomes will include histologic assessment of intestinal morphology.

Clinical Observations: The clinical scoring of NEC will be accomplished via the system described by Thymann et al. and used to generate preliminary data. The piglets will be monitored every 3 hours for intestinal disease (stool color and consistency, abdominal distention, pain at palpation, and respiratory distress). At tissue collection, the gastrointestinal track will be placed on an ice-cold surface, opened, and cleared of its contents. Macroscopic scoring of NEC will be examined in the stomach, 3 regions of the small intestine, and the colon. Each section will be given a score of 1-5 based on the extent of edema, hemorrhage, necrosis, and/or pneumatosis. Presence of NEC will be defined as a score of greater than 3 in any section.

Histologic Assessments: The small intestine will be divided into thirds and 2-5 cm non-necrotic sections from the middle of each third excised, fixed, and embedded. A portion of the stomach and colon will be similarly treated. Each fixed portion of tissue will be sectioned into 3-5 μm sections and placed on glass slides. A section of each tissue block will be stained with H&E. Then histologic scoring of NEC will be determined on each section as well as measuring villus height and crypt depth on 10 representative villi from a section from each third.

Tissue samples will also be examined immunohistochemically for the presence of SOX9 (a marker of intestinal stem cell proliferation), cleaved caspase 3 (CC3; a marker of cell death), and Ki-67 (a marker of cell proliferation). Immunohistochemistry will be used to quantify changes in the number of specific cell types. Heat-induced epitope retrieval will be performed by placing slides into citrate target retrieval solution for 30 seconds at 120° C. followed by 90° C. for 10 seconds in a pascal pressure chamber. Slides will be cooled to room temperature and moved to a slide stainer after which they will be incubated in a peptide-blocking agent for 30 minutes. Primary antibodies previously shown to positively identify stem and progenitor cells in normal porcine tissue will be used. Primary antibodies will be applied to tissue sections diluted in common antibody diluent (BioGenex). Rabbit α-SOX9 (EMD Millipore) will be diluted 1:200, mouse α-Ki-67 diluted to 1:500, and aCC3 (rabbit, 1:400). Primary antibody incubation will be performed for 30 minutes at room temperature. Slides will be incubated for 30 minutes in a 1:1 dilution of ImmPRESS™ polymer anti-rabbit IgG reagent followed by 30 minutes in ImmPact DAB peroxidase substrate solution.

For each slide evaluated, only well-oriented crypts will be used to obtain cell counts. A well-oriented crypt will be identified as one with a crypt base closely apposed to the muscularis mucosa layer and that may be extended and opened fully into the gut lumen. At least 10 well-oriented crypts for each protein biomarker will be imaged, and positive cells per crypt counted and averaged as previously described. These markers will provide a survey of stem cell health within the crypts as well as apoptosis and proliferation within the villus. Customary pathology organs (heart, lungs, liver, kidneys) will be observed, fixed, and sectioned for signs of toxicity.

Statistical Analyses: To ensure rigor and reproducibility of the proposed studies, the study will (1) randomize the subject animals within each test group; (2) have negative and positive controls in all experiments; (3) conduct blinded analysis of specimens; and (4) use appropriate tests to determine significance of differences between experimental groups. Group differences of NEC incidence will be evaluated by Fisher's exact test. Lesions scores on the gastrointestinal tract will be analyzed by a nonparametric Kruskal-Wallis test. Quantitative data on proliferation, villus height, and crypt depth will be analyzed via a 2-way ANOVA to determine the effect of dose on the animals. A Tukey post hoc test will be performed to identify differences between specific groups.

The primary outcome of this experiment may be a reduction in clinical NEC within the acellular placental extract treated animals. Preliminary studies of acellular placental extract in this model showed no clinical signs of NEC. The study is powered to demonstrate a 75% reduction in NEC incidence with 95% power and an α=0.05. Based on the in vitro data presented (FIGS. 1-3), observations may include an increase in SOX9 within the crypts, a decrease in cleaved Caspase 3 and an increase in Ki-67 in acellular placental extract treated animals. This may demonstrate an increased stem cells, decreased apoptosis, and increased proliferation in response to acellular placental extract. The potential problems to be encountered lay primarily within the execution of the in-life portion of the animal study. The study involves placing as many as 18 piglets into what is essentially neonatal intensive care for 96 hours at the same time. Based on preliminary studies, endotracheal tube size, resuscitation medicines (e.g. Narcan), and optimal order of instrumentation were issues. The immunohistochemical analysis of the tissue may be difficult. Limited antibodies available for these markers in porcine tissue. However, these techniques are performed and optimized for observing these markers together.

Specific Aim 3. To evaluate the safety of orally administered acellular placental extract in a standard toxicology testing assessment in newborn rodents.

Rationale: Acellular placental extract is a complex mixture of chemokines. A dosing range juvenile toxicology study is proposed for this aim. Sprague-Dawley rats will be administered acellular placental extract via gavage feeding beginning on day 4 of life with escalating doses (low, target, and 2 higher doses) beginning at 1 mg/mL total protein and increasing to 20× to assess possible toxicity. Dosing will continue through day 18. In-life assessment of growth will be monitored via body weight, tibial length, and crown to tail measurements repeated bi-weekly on each animal. Toxicity will be assessed with histology of the heart, lung, liver, kidney, and brain. Human proteins within the serum of the rats will be measured at 6 intervals. Male and female rats will be used in this objective to allow comparison of sex differences in potency, toxicity, and kinetics. These tests will evaluate any clinical or histological toxicity generated by acellular placental extract treatment.

Experimental Design: The goal of this study is to assess the toxicity of acellular placental extract. The vehicle (normal saline) will also be tested to establish baseline toxicity. Rats will be euthanized at 6 time-points during the study and at day 19. Blood will be collected from all rats on the day of euthanasia, followed by a comprehensive necropsy. Blood and tissues will be evaluated for abnormalities.

Amniotic fluid is the material that stimulated development of the fertilized egg to a term infant in approximately 260 days. Placental tissue may be inherently immune-privileged. Amniotic-based products have been used as human therapies for over a century with minimal adverse events. Thus, no adverse events are expected in the toxicology endpoints to be monitored in this study. Quantification of human proteins in the serum of the rats should not present any issues. However, serum from the control group and pretreatment serum is being collected to observe any interference of rat proteins in the human protein ELISA. The results from these samples will be used to determine and subtract any background observed.

### Vertebrate Animals—Description of Procedures

### Specific Aims 1

Male and female 1-2 day old piglets will be used to obtain tissue for stem cell culture in experiments proposed in Specific Aim 1. Piglets will be heavily sedated and then euthanized immediately for tissue collection. The study group estimates for Aim 1 that 6 animals will be used over the year including control animals based on preliminary data used to calculate a power analysis. In Aim 1, male and female 1-2 day old Yorkshire Cross littermate pigs will be used. Euthanasia (pentobarbital overdose; IV) will be performed following sedation. Tissue will be immediately collected from the ileum.

### Specific Aims 2

The premature piglet model will be conducted under an IACUC-approved protocol. After preterm piglets are delivered via cesarean section, they will be resuscitated and initial O₂ saturations and heart rates measured with a pulse oximeter. Resuscitation will include: suctioning the nasopharynx free of amniotic fluid, receiving intermittent bag-mask ventilation with oxygen to aid with respiration, and placement in temperature-regulated incubators maintained at 31-32° C. Doxapram hydrochloride will be provided sublingually or via an umbilical vessel to newborn pigs that do not ventilate adequately. Once the piglets are stabilized, they will have an orogastric feeding tube placed, if not already placed, and a catheter placed in the umbilical artery to facilitate oral gastric feeding, total parenteral nutrition (TPN) feeding, and the administration of the test compound over a 96-hour study period. The orogastric feeding tube (5 or 8 French size) will be introduced via a small incision made in the cheek, behind the teeth (to prevent piglets from chewing the tube), and passed into the stomach or distal esophagus. If not already placed, a sterile catheter will then be placed in the umbilical artery and advanced 17-20 cm into the dorsal thoracic aorta and will be secured to the cord stump by two or more ligatures. Sterile maternal plasma (10 to 20 mL/kg maternal plasma) will be transfused to each piglet to provide passive immunity. The piglets will receive supplemental oxygen during the first 48 hours and will gradually be weaned off supplemental oxygen by 72 hours post birth. All piglets will be housed individually in warmed cages serving as incubators at an ambient temperature of 31-32 ° C.

All piglets will be weighed at birth and randomly assigned to the control or study group in a 1:3 ratio. The control group (n=11), acellular placental extract 4 mg/ml protein: (n=12), acellular placental extract 8 mg/ml protein: (n=12) and acellular placental extract 20 mg/ml protein (n=33). Stratification of groups will be based upon weight and initial O₂ saturations. Once all of the piglets are removed from the uterus of the sow, as much maternal blood as possible will be collected from one of the uterine veins using 60 cc syringes while the sow is in a deep plane of surgical anesthesia. Once the blood is collected, the sow will be euthanized with pentobarbital drug at 110 mg/kg IV while still under anesthesia.

The control group will receive parenteral followed by oral feedings. Oral feeds will start at 48 hours after delivery and may be given as a bolus once every 3 hours or as a continuous drip that achieves 15 ml/kg every 3 hours to meet the daily caloric needs. The piglets will first receive TPN by continuous infusion via an arterial catheter for 48 hours using Kabiven portfolio (Fresenius Kabi USA, LLC.), starting at 4 mL/kg for the first 24 hours and advancing to 6 mL/kg/hour, and adjusted to meet their nutritional requirements (see Bjornvad 2008).

After 48 hours of TPN, the piglets will only receive oral feedings for 3 days before euthanasia and tissue collection. During the first 6 hours of enteral feeding, all piglets will receive formula (80 g Pepdite, 70 g Maxipro, and 75 g Liquigen per liter of water. In addition, lactated Ringer's solution will be given by orogastric tube at a rate of 2.5 mL/kg/hr for the first 24 hours post-delivery, then increasing to 5 mL/hr/kg through 48 hours post-delivery. At that point, piglets will be switched to 5 mL/kg/hour of formula as described by Bjornvad (2008).

Treatment groups will have arterially delivered TPN and orally delivered acellular placental extract followed by oral feedings. Oral feedings may be given as a bolus once every 3 hours or as a continuous drip that achieves the 15 ml/kg every 3 hours to meet piglets' daily caloric needs. The piglets will receive TPN by arterial catheter for 48 hours using Kabiven portfolio (Fresenius Kabi USA, LLC.) starting at 4 mL/kg for the first 24 hours and advancing to 6 mL/kg/hour adjusted in composition to meet the nutritional requirements of the preterm pigs. After 2 days of TPN and oral acellular placental extract, the piglets will only receive oral feedings for 3 days before euthanasia and tissue collection. During the first 6 hours of enteral feeding, all piglets will receive formula (80 g Pepdite, 70 g Maxipro, and 75 g Liquigen per liter of water). In addition to TPN, acellular placental extract will be administered via the orogastric tube within 3 hours of birth. Acellular placental extract will be dissolved in lactated Ringer's solution to a concentration of 4 mg/mL total protein and administered at a rate of 2.5 mL/kg/hr for the first 24 hours post-delivery, then increasing to 5 mL/hr/kg through 48 hours post-delivery. At that point, piglets will be switched to 5 mL/kg/hour of formula as described by Bjornvad.

To compensate for immature lung function, 1 to 2 L/min of 100% humidified oxygen will be introduced into the incubator environment for the first 6 to 12 hours after birth. Thereafter, supplemental humidified oxygen will be provided only to piglets showing signs of respiratory distress.

Monitoring: All animals will be monitored every 15-20 minutes for the first 2 days. They will be monitored every 30 minutes for the next 3 days. Vital signs along with mucus membrane and capillary refill time will be recorded every hour with complete physical exams every 3 hours. Oxygen saturation and heart rate will be measured by pulse oximetry every hour.

Early endpoints include decreased activity level, feeding intolerance, abdominal distention, vomiting, diarrhea, and bloody stool. Piglets that do not improve within 6 hours of onset of symptoms will be humanely euthanized and a necropsy performed. Physical examinations and assessment of each piglet will be performed every 3 hours prior to each feeding. Piglets with heart rates ranging from 163-175 bpm with a corresponding SpO₂ of 70% or less will be immediately assessed and euthanized if necessary. In previous studies, animals with fulminating NEC had heart rates of 163-175 bpm, decreasing O₂ saturations of less than 87% and lower, and falling body temperatures were not likely to survive.

Necropsy: On day 5, all piglets will be euthanized with a pentobarbital-based euthanasia solution at a dose of at least 110 mg/kg given through the umbilical catheter. Once the animal is deceased, a necropsy will be performed. The gastrointestinal tract will be removed and opened to determine the presence of NEC. The following clinical NEC scoring system will be applied to characterize the extent to which damage as indicated by macroscopic evidence of inflammation, edema, hemorrhage, necrosis, and pneumatosis intestinalis occurred in the stomach, small intestine, and/or colon: 1. no or minimal focal hyperemic gastroenterocolitis; 2. mild focal gastroenterocolitis; 3. moderate locally extensive gastroenterocolitis; 4. severe locally extensive hemorrhagic gastroenterocolitis; 5. severe locally extensive hemorrhagic and necrotic gastroenterocolitis; and 6. severe diffuse hemorrhagic and necrotic gastroenterocolitis.

An NEC score of 3 or more for one gastrointestinal section will be considered positive for NEC. Sections of the gastrointestinal tract from all piglets will be placed into buffered formalin for microscopic evaluation and grading.

This aim is designed in 2 phases: to demonstrate a 75% reduction in the primary outcome of NEC incidence, and to show a dose effect with acellular placental extract treatment. A total of 6 sows and 72 piglets is being requested. The efficacy portion of this aim will require 48 pre-mature piglets. Published results using the proposed model demonstrate approximately a 50% incidence of NEC when given the standard of care (48 hours of TPN nutrition via umbilical vein followed by enteral feeds for 2 days). The animal numbers requested provide for 95% power and 0.05 alpha to demonstrate a 75% reduction in the primary outcome, NEC incidence. A 1:3 sampling scheme of standard of care to acellular placental extract intervention will yield n=11 controls and n=33 treatment group. The study will allow for an extra 4 piglets are planned, in the same 1:3 scheme, in case any piglets must be removed from the study, for a total of 48 piglets in the efficacy study. The remaining 24 pre-mature piglets (n=12/group) will be equally divided between the 2× and 5×acellular placental extract arms for the dose escalation portion of the aim. This is sufficient to determine a change of 7.5% in the NEC incidence of either escalated dose compared to the expected outcome of the treatment group in the efficacy portion (16.5%), with a power of 80% and an alpha of 0.05.

Acellular Placental Extract Preparation: The placenta and amniotic membrane are procured by a placenta donation coordinator employed by the tissue bank. The coordinator/QA department screens the donor as described above. The coordinator places the placenta/amniotic membrane in 0.9% normal saline. Following transfer to Plakous, the placental disc is separated from the amniotic membrane, chorion, and umbilical cord by gross tissue homogenization. Placental disc pieces are then grossly homogenized using a laboratory blender with PBS that also contains protease inhibitor (PI). The gross homogenate is then centrifuged to separate bloody fluid (discarded). The blended tissue forms a pellet at the bottom of the container and is then re-suspended in PBS with PI at a ratio of ≈1:5 tissue volume: total volume. The mixture is cooled to 4° C. and cell lysis is accomplished by high-pressure homogenization. Cellular debris is removed by centrifugation and filter-sterilized. The extract is then freeze-dried and placed in sterile vials.

Quality Control: Bioactivity Testing. Following freeze drying, each batch of extract is tested for bioactivity. A fibroblast proliferation assay is currently used to determine bioactivity. Current pass/fail is determined as significantly different proliferation from the control of fetal bovine serum over 48 hours of treatment. Methods developed in Aim 1 of this proposal will be validated to replace this fibroblast assay as the bioactivity assay for the NEC product clearance. In vitro results of chondrocyte assays have been presented. These data were generated from 3 distinct batches, both individual placenta preparation and pooled placenta batches.

Component Concentration Consistency Testing. Total protein (mg/ml) is planned as a release criterion for acellular placental extract. Since the product contains chemokines, cytokines, and growth factors, the study will quantify 6 specific proteins (IL-10, IL-4, EGF, FGF, GRO-α, and SDF-1) implicated in preventing NEC and intestinal development in pre-clinical experiments with a multiplex assay. This approach is similar to that for other biologic therapeutics, including IVIg, pooled fresh frozen plasma, and therapeutics being developed from the secretome of cultured, non-adherent placenta cells.

### Illustrative Embodiments of Suitable Compositions, Fibers, Composites, Products

As used below, any reference to formulations or methods for intestinal conditions is to understood as a reference to each of the those formulations or methods disjunctively (e.g., “Illustrative embodiments 1-4 is to be understood as illustrative embodiment 1, 2, 3, or 4”).

Illustrative embodiment 1 is a formulation comprising: an acellular extract of placental tissue and a solvent.

Illustrative embodiment 2 is the formulation of any preceding or subsequent illustrative embodiment, wherein the placental tissue comprises a placental disc.

Illustrative embodiment 3 is the formulation of any preceding or subsequent illustrative embodiment, wherein the placental tissue further comprises a villous chorion.

Illustrative embodiment 4 is the formulation of any preceding or subsequent illustrative embodiment, wherein the solvent comprises milk, water, saline, a sodium lactate solution, or a sugar solution.

Illustrative embodiment 5 is the formulation of any preceding or subsequent illustrative embodiment, wherein the milk is natural mammalian milk or synthetic mammalian milk.

Illustrative embodiment 6 is the formulation of any preceding or subsequent illustrative embodiment, wherein the milk is dehydrated.

Illustrative embodiment 7 is the formulation of any preceding or subsequent illustrative embodiment, further comprising a diluent, an excipient, or a carrier.

Illustrative embodiment 8 is the formulation of any preceding or subsequent illustrative embodiment, further comprising one or more exogenous protease inhibitors.

Illustrative embodiment 9 is the formulation of any preceding or subsequent illustrative embodiment, wherein the one or more exogenous protease inhibitors comprises 4-(2-Aminoethyl) benzenesulfonyl fluoride hydrochloride (AEBSF), Bovine Lung Aprotinin, N-(trans-Epoxysuccinyl)-L-leucine 4- guanidinobutylamide, Leupeptin, N-ethylmaleimide (NEM), phenylmethylsulfonylfluoride (PMSF), ethylenediamine tetraacetic acid (EDTA), ethylene glycol-bis-(2-aminoethyl(ether)NNN′N′-tetraacetic acid, ammonium chloride, boceprevir, danoprevir, narlaprevir, telaprevir, or vaniprevir.

Illustrative embodiment 10 is the formulation of any preceding or subsequent illustrative embodiment, further comprising vitamins or minerals.

Illustrative embodiment 11 is the formulation of any preceding or subsequent illustrative embodiment, further comprising antibiotics.

Illustrative embodiment 12 is the formulation of any preceding or subsequent illustrative embodiment, wherein the acellular extract comprises one or more proteins.

Illustrative embodiment 13 is the formulation of any preceding or subsequent illustrative embodiment, wherein a concentration of the one or more proteins in the oral formulation is up to 25 mg/mL.

Illustrative embodiment 14 is the formulation of any preceding or subsequent illustrative embodiment, wherein a concentration of the one or more proteins in the oral formulation is up to 15 mg/mL.

Illustrative embodiment 15 is the formulation of any preceding or subsequent illustrative embodiment, wherein a concentration of the one or more proteins in the oral formulation is from 2 mg/mL to 10 mg/mL.

Illustrative embodiment 16 is the formulation of any preceding or subsequent illustrative embodiment, wherein the acellular extract is suspended in the solvent.

Illustrative embodiment 17 is the formulation of any preceding or subsequent illustrative embodiment, wherein the acellular extract is dissolved in the solvent.

Illustrative embodiment 18 is the formulation of any preceding illustrative embodiment, wherein the formulation is administered orally.

Illustrative embodiment 19 a method for preventing or reducing symptoms of an intestinal condition in a subject, the method comprising: obtaining a liquid formulation comprising an acellular extract of placental tissue and a solvent; and delivering a therapeutically effective amount of the liquid formulation to a gastric system of the subject.

Illustrative embodiment 20 is the method of any preceding or subsequent illustrative embodiment, wherein the intestinal condition comprises Necrotizing Enterocolitis (NEC).

Illustrative embodiment 21 is the method of any preceding or subsequent illustrative embodiment, wherein the subject is a premature infant born at 37 weeks gestation or less.

Illustrative embodiment 22 is the method of any preceding or subsequent illustrative embodiment, wherein the premature infant has a birthweight of 1500 g or less.

Illustrative embodiment 23 is the method of any preceding or subsequent illustrative embodiment, wherein the delivery of the liquid formulation is oral.

Illustrative embodiment 24 is the method of any preceding or subsequent illustrative embodiment, wherein the delivery of the liquid formulation is by a percutaneous feeding tube.

Illustrative embodiment 25 is the method of any preceding or subsequent illustrative embodiment, wherein the liquid formulation reduces inflammation in the intestine of the subject.

Illustrative embodiment 26 is the method of any preceding or subsequent illustrative embodiment, wherein a villous height of the intestine of the subject is increased.

Illustrative embodiment 27 is the method of any preceding or subsequent illustrative embodiment, wherein a crypt depth of the intestine of the subject is increased.

Illustrative embodiment 28 is the method of any preceding or subsequent illustrative embodiment, wherein the liquid formulation is delivered to the subject continuously for up to 30 days.

Illustrative embodiment 29 is the method of any preceding or subsequent illustrative embodiment, wherein the liquid formulation is delivered to the subject continuously for up to 10 days

Illustrative embodiment 30 is the method of any preceding or subsequent illustrative embodiment, wherein the liquid formulation is delivered to the subject contemporaneous with periodic feedings of the subject.

Illustrative embodiment 31 is the method of any preceding or subsequent illustrative embodiment, wherein the solvent comprises milk, water, saline, a sodium lactate solution, or a sugar solution.

Illustrative embodiment 32 is the method of any preceding illustrative embodiment, wherein a concentration in the liquid formulation of one or more proteins from the acellular extract is up to 25 mg/mL.

The foregoing description of certain examples, including illustrated embodiments, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

1. An oral formulation comprising:
an acellular extract of placental tissue comprising a placental disc; and
a solvent.

2. (canceled)

3. The oral formulation of claim 1, wherein the placental tissue further comprises a villous chorion.

4. The oral formulation of claim 1, wherein the solvent comprises natural mammalian milk, synthetic mammalian milk, water, saline, a sodium lactate solution, a sugar solution, or combinations thereof.

5. (canceled)

6. The oral formulation of claim 4, wherein the milk is dehydrated.

7. The oral formulation of claim 1, further comprising a diluent, an excipient, or a carrier.

8. The oral formulation of claim 1, further comprising one or more exogenous protease inhibitors.

9. The oral formulation of claim 8, wherein the one or more exogenous protease inhibitors comprises 4-(2-Aminoethyl) benzenesulfonyl fluoride hydrochloride (AEBSF), Bovine Lung Aprotinin, N-(trans-Epoxysuccinyl)-L-leucine 4- guanidinobutylamide, Leupeptin, N-ethylmaleimide (NEM), phenylmethylsulfonylfluoride (PMSF), ethylenediamine tetraacetic acid (EDTA), ethylene glycol-bis-(2-aminoethyl(ether)NNN′N′-tetraacetic acid, ammonium chloride, boceprevir, danoprevir, narlaprevir, telaprevir, or vaniprevir.

10. The oral formulation of claim 1, further comprising a vitamin, a mineral, or combinations thereof.

11. The oral formulation of of claim 1, further comprising an antibiotic.

12. The oral formulation of claim 1, wherein the acellular extract comprises up to 25 mg/mL of one or more proteins.

13-17. (canceled)

18. A method for preventing or reducing symptoms of an intestinal condition in a subject, the method comprising:
obtaining a liquid formulation comprising an acellular extract of placental tissue and a solvent; and
delivering a therapeutically effective amount of the liquid formulation to a gastric system of the subject.

19. The method of claim 18, wherein the intestinal condition comprises Necrotizing Enterocolitis (NEC).

20. The method of claim 18, wherein the subject is a premature infant born at 37 weeks gestation or less.

21. The method of claim 20, wherein the premature infant has a birthweight of 1500 g or less.

22. The method of claim 1, wherein the delivery of the liquid formulation is oral.

23. The method of claim 1, wherein the delivery of the liquid formulation is by a percutaneous feeding tube.

24. The method of claim 1, wherein the liquid formulation reduces inflammation in the intestine of the subject, increases a villous height of the intestine of the subject, increases a crypt depth of the intestine of the subject, or combinations thereof.

25-26. (canceled)

27. The method of claim 1, wherein the liquid formulation is delivered to the subject continuously for up to 30 days.

28. The method of claim 1, wherein the liquid formulation is delivered to the subject contemporaneous with periodic feedings of the subject.

29. The method of of claim 1, wherein the solvent comprises milk, water, saline, a sodium lactate solution, or a sugar solution.

30. (canceled)
